(19) United States
(12) Patent Application Publication
(10) Pub. No.: US 2025/0109382 A1
(43) Pub. Date: Apr. 3, 2025

(54) METHOD FOR PRODUCING MEMORY-LIKE NATURAL KILLER CELLS AND ANTICANCER USE OF MEMORY-LIKE NATURAL KILLER CELLS PRODUCED THEREBY

(71) Applicant: ELPHIS CELL THERAPEUTICS, Seoul (KR)

(72) Inventor: Seonmin HONG, Yongin-si (KR)

(73) Assignee: ELPHIS CELL THERAPEUTICS, Seoul (KR)

(21) Appl. No.: 18/857,913

(22) PCT Filed: Apr. 20, 2023
(86) PCT No.: PCT/KR2023/005351

§ 371 Date: Oct. 18, 2024

(30) Foreign Application Priority Data
Apr. 21, 2022 (KR) 10-2022-0049661

(51) Int. Cl. C12N 5/0783; A61K 35/17; A61K 45/06; A61P 35/00

(52) U.S. Cl. CPC C12N 5/0646; A61K 35/17; A61K 45/06; A61P 35/00; C12N 2501/2302; C12N 2501/2312; C12N 2501/2315; C12N 2501/2318; C12N 2533/52

(57) ABSTRACT

The present invention relates to a method for producing memory-like natural killer (NK) cells and anticancer use of memory-like NK cells produced thereby. The method for producing memory-like NK cells, according to the present invention, is an optimal autoimmune cell culture method for producing memory-like NK cells with an increased ability to kill cancer cells while the proliferation thereof is maintained. Memory-like NK cells produced thereby have an excellent ability to kill cancer cells compared to NK cells, have a memory-like ability, thus having the ability to be activated again and proliferate again when a second stimulus is applied, and has the advantage of being able to survive for 2 months or longer in vivo, and thus can be used as an immune cell therapeutic agent or for the prevention or treatment of cancer.

### TECHNICAL FIELD

The present invention relates to a method for producing memory-like natural killer (NK) cells and anticancer use of memory-like NK cells produced thereby.

### BACKGROUND ART

Natural killer cells (hereinafter NK cells), which belong to lymphocytes in a human immune system, are responsible for innate immunity and play a role in quickly recognizing and eliminating abnormal cells such as external pathogens or internal cancer cells. In particular, in order to activate T cells responsible for acquired immunity, a complex process is essential in which antigen-presenting cells need to first degrade target cells and present antigens to MHC, whereas the NK cells receive attention as an immune cell therapeutic agent in areas that do not require the presenting of the antigen. Meanwhile, the NK cells are a subtype of lymphocytes present in bone marrow, lymph nodes, and peripheral blood, and account for approximately 10 to 15% of all lymphocytes and are the third most common subtype after T cells and B cells. As immune cells with an ability to kill cancer cells or virus-infected cells, the NK cells play an important role in innate immune responses. The function of the NK cells is regulated by the interaction between cell surface receptors and corresponding target cell ligands without stimulation of specific antigens, and these receptors are largely divided into an activating receptor and an inhibitory receptor. As representative activating receptors that control the function of NK cells by transmitting activation signals to NK cells, natural cytotoxicity receptors (NCRs; NKp30, NKp44, and NKp46), NKG2D, etc. are well known. The NK cells detect target cells through various activating receptors with different ligand specificities, and these receptors mainly induce activation through phosphorylation, and each signaling characteristic is very different. The NCRs (NKp30, NKp44, NKp46) and CD16 are known to form complexes with FcRγ, CD3γ, and DAP12 having ITAM motifs to transmit activation signals similar to the TCR and BCR of T cells and B cells. NKG2D binds to a DAP10 adapter having a YINM motif and transmits signals through a PI3K or Grb2-Vav1 complex. In particular, it has been shown that NK cells with high expression of NCRs have higher cytotoxicity than cells with low expression, so that the activation level of NK cells may be confirmed depending on the expression level of NCRs. Activated NK cells may destroy target cells by synthesizing various granules and secreting the granules out of the cells. Among the representative proteins contained in the granules, Perforin and Granzyme play a major role in destroying target cells. The Perforin gathers on the cell membrane of the target cell to form a complex and punctures the cell membrane to cause cell lysis, and the Granzyme is introduced into the cells through the holes generated in the cells to not only activate caspase, but also cause apoptosis through various mechanisms.

Meanwhile, T cells recognize antigens presented on MHC and also distinguish between self cells and non-self cells. When the non-self cells are recognized, the non-self cells are recognized as abnormal cells and immediately killed, so that cancer cells have significantly reduced MHC expression on the surface to evade the immune system. However, when the NK cells recognize the MHC of other cells, an inhibitory signal is triggered, and when there is no MHC of the target cell, an activation signal is rather triggered to eliminate the target cells. Accordingly, the NK cells are slightly more advantageous than T cells in terms of the ability to kill cancer cells. In addition, the NK cells induce apoptosis of cancer cells by binding to the death receptors of cancer cells using death ligands such as FasL and TRAIL. The binding of these receptors activates caspase-8 and -10 in cancer cells to activate caspase-3 and -7, thereby causing apoptosis. In addition, the NK cells secrete various cytokines and chemokines and play a bridgehead role in activating adaptive immune responses through direct interaction with antigen-presenting cells. IFN-α secreted from activated NK cells is known to play an important role in increasing innate immune responses to pathogens by activating and maturing monocytes and dendritic cells and play an important role in the early stages of Th1 immune responses.

In order for the NK cells to have the ability, differentiation priming is required before meeting target cells. Accordingly, precursor NK cells isolated from mice and humans have significantly reduced cancer cell killing ability and IFN-γ production ability. IL-2, IL-15, etc. have been partially reported as differentiation-priming cytokines that may maximize the ability of these NK cells. For example, it has been reported that IL-2 had a function of enhancing the proliferation and activation of mature NK cells. In addition, IL-15 is known to be involved in NK cell differentiation. In addition, IL-21 is a cytokine secreted by activated CD4+ T cells, and a IL-21 receptor (IL-21R) is known to be expressed on lymphocytes such as dendritic cells, NK cells, T cells, and B cells.

In the case of cancer patients, since the overall immune system is not functioning properly, the ability to kill the cancer cells is not sufficient using the immune cells in the body as they are. Recently, an immune cell therapeutic agent has been actively developed in which NK cells are isolated and cultured from the peripheral blood of a patient to activate the ability to kill cancer cells, and then injected back into the patient. However, since general NK cells enter the bone marrow and disappear after 1 to 2 weeks of injection into the body, NK cell therapeutic agents that have been developed so far require NK cells to be injected into the patient once every 1 to 2 weeks, which is a burden on patients. Accordingly, recently, when the NK cells were cultured in a resting phase after initial priming and then re-primed, it has been confirmed to produce NK cells that have memory-like cell functions like T cells, have good cancer cell killing ability, and are survivable for a long time in the body, and thus related research has been actively conducted. However, it is difficult to develop therapeutic agents/therapy due to the disadvantages of not proliferating well during the production process and being a burden on patients and donors because a large amount of blood is required to secure a sufficient number of NK cells (Matteo Tanzi, et al., Cancers, 13:1577, 2021).

Therefore, there is a need to produce more active memory-like NK cells instead of low-active NK cells existing in the body and to use the NK cells for anti-cancer use, and the like.

### DISCLOSURE

### Technical Problem

An object of the present invention is to provide a method for producing memory-like NK cells.

In addition, another object of the present invention is to provide memory-like NK cells.

In addition, yet another object of the present invention is to provide a pharmaceutical composition for preventing or treating cancer.

In addition, yet another object of the present invention is to provide an immune cell therapeutic agent for treating cancer.

In addition, yet another object of the present invention is to provide an anticancer adjuvant for enhancing the activity of an anticancer agent.

In addition, yet another object of the present invention is to provide a method for preventing or treating cancer.

### Technical Solution

An aspect of the present invention provides a method for producing memory-like NK cells.

In addition, another aspect of the present invention provides memory-like NK cells produced by the method.

In addition, yet another aspect of the present invention provides a pharmaceutical composition for preventing or treating cancer including the memory-like NK cells as an active ingredient.

In addition, yet another aspect of the present invention provides an immune cell therapeutic agent for treating cancer including the memory-like NK cells as an active ingredient.

In addition, yet another aspect of the present invention provides an anticancer adjuvant for enhancing the activity of an anticancer agent including the memory-like NK cells as an active ingredient.

In addition, yet another aspect of the present invention provides a method for preventing or treating cancer including administering the pharmaceutical composition or the cell therapeutic agent to a subject.

### Advantageous Effects

According to the present invention, the method for producing memory-like NK cells is an optimal autoimmune cell culture method for producing memory-like NK cells with an increased ability to kill cancer cells while the proliferation thereof is maintained. Memory-like NK cells produced thereby have an excellent ability to kill cancer cells compared to NK cells, have a memory-like ability, thus having the ability to be activated again and proliferate again when a second stimulus is applied, and has the advantage of being able to survive for 2 months or longer in vivo, and thus can be used as an immune cell therapeutic agent or for the prevention or treatment of cancer.

### DESCRIPTION OF DRAWINGS

FIG. 1 is a diagram of measuring the purity (distribution) of NK cells according to an embodiment of the present invention:
Control: Negative control cells in which peripheral blood mononuclear cells are cultured in a culture medium containing 2 to 10% autologous plasma and 1 ng/ml of IL-15; and
Memory-like NK cells: Memory-like NK cells produced by the method of the present invention.

FIG. 2 is a diagram of confirming the activity (ability to kill cancer cells) of memory-like NK cells according to an embodiment of the present invention:
Control: Negative control cells in which peripheral blood mononuclear cells are cultured in a culture medium containing 2 to 10% autologous plasma and 1 ng/ml of IL-15; and
Memory-like NK cells: Memory-like NK cells produced by the method of the present invention.

FIG. 3 is a diagram of analyzing the cell surface expression of memory-like NK cell-specific markers according to an embodiment of the present invention:
Control: Negative control cells in which peripheral blood mononuclear cells are cultured in a culture medium containing 2 to 10% autologous plasma and 1 ng/ml of IL-15; and
Memory-like NK cells: Memory-like NK cells produced by the method of the present invention.

FIG. 4 is a diagram of confirming the cumulative proliferation rate by comparing the total number of cells finally harvested with the total number of cells at the beginning of the culture, according to an embodiment of the present invention.

FIG. 5 is a diagram of comparing the overall cell proliferation rate of an experimental group (memory-like NK cells) with a negative control according to an embodiment of the present invention:
Control: Negative control cells in which peripheral blood mononuclear cells are cultured in a culture medium containing 2 to 10% autologous plasma and 1 ng/ml of IL-15; and
Memory-like NK cells: Memory-like NK cells produced by the method of the present invention.

FIG. 6 is a diagram of comparing the cell proliferation rate by confirming the number of NK cells finally produced compared to the number of NK cells at the beginning of the culture according to an embodiment of the present invention.

FIG. 7 is a diagram of comparing final NK cell proliferation rate compared to primary cultured NK cells in an experimental group (memory-like NK cells) with a negative control according to an embodiment of the present invention:
Control: Negative control cells in which peripheral blood mononuclear cells are cultured in a culture medium containing 2 to 10% autologous plasma and 1 ng/ml of IL-15; and
Memory-like NK cells: Memory-like NK cells produced by the method of the present invention.

### BEST MODE OF THE INVENTION

Hereinafter, embodiments of the present invention will be described in detail with reference to the accompanying drawings. However, the following embodiments are presented as examples for the present invention, and when it is determined that a detailed description of well-known technologies or configurations known to those skilled in the art may unnecessarily obscure the gist of the present invention, the detailed description thereof may be omitted, and the present invention is not limited thereto. Various modifications and applications of the present invention are possible within the description of claims to be described below and the equivalent scope interpreted therefrom.

Terminologies used herein are terminologies used to properly express preferred embodiments of the present invention, which may vary according to a user, an operator's intention, or customs in the art to which the present invention pertains. Therefore, these terminologies used herein will be defined based on the contents throughout the specification. Throughout the specification, unless explicitly described to the contrary, when a certain part “comprises” a certain component, it will be understood to imply the inclusion of stated elements but not the exclusion of any other elements.

In an aspect, the present invention relates to a method for producing memory-like NK cells comprising 1) isolating peripheral blood mononuclear cells (PBMC) from peripheral blood: 2) treating and culturing plasma, anti-NKp46 antibody, IL-2, and IL-18:3) transferring the cells to a new culture vessel and treating and culturing the cells with plasma and IL-2:4) transferring the cells to a new culture vessel and treating and culturing the cells with plasma, IL-12, IL-15, and IL-18; and 5) transferring the cells to a new culture vessel and treating and culturing the cells with plasma, IL-12, and IL-15.

In an embodiment, in step 2), the cells may be cultured in a culture vessel coated with fibronectin, γ-globulin, and anti-CD56 antibody.

In an embodiment, in step 2), the cells may be treated with 1 to 20% (w/w) of the plasma, 0.01 to 10 μg/ml of the anti-NKp46 antibody, 10 to 5000 IU/ml of IL-2, and 2 to 500 ng/ml of IL-18.

In an embodiment, step 2) may be performed twice or more, and the cells are primarily treated with the plasma, the anti-NKp46 antibody, IL-2, and IL-18 and cultured until the density of the cells reaches 80% or more, and then secondarily treated with the plasma, the anti-NKp46 antibody, IL-2, and IL-18 and cultured until the density of the cells reaches 80% or more.

In an embodiment, in step 3), the cells may be treated with 1 to 20% (w/w) of the plasma and 10 to 5000 IU/ml of IL-2.

In an embodiment, step 3) may be performed twice or more, and the cells are primarily treated with the plasma and IL-2 and cultured until the density of the cells reaches 80% or more, and then secondarily treated with the plasma and IL-2 and cultured until the density of the cells reaches 80% or more.

In an embodiment, in step 4), the cells may be treated with 1 to 20% (w/w) of the plasma, 0.5 to 200 ng/ml of IL-12, 0.1 to 100 ng/ml of IL-15, and 2 to 500 ng/ml of IL-18.

In an embodiment, the culturing in step 4) may be performed for 10 to 20 hours, and more preferably performed for 12 to 16 hours.

In an embodiment, in step 5), the cells may be treated with 1 to 20% (w/w) of the plasma, 10 to 5000 IU/ml of IL-2, and 0.1 to 100 ng/ml of IL-15.

In an embodiment, after step 5), the method may further include treating and culturing the cells with a fresh medium containing IL-2, and 10 to 5000 IU/ml of IL-2 may be treated.

In an embodiment, the plasma may be plasma isolated from the peripheral blood, and the peripheral blood mononuclear cells (PBMC) and plasma may be autologous to the subject.

The method for producing the memory-like NK cells of the present invention is an optimal autoimmune cell culture method for preparing/producing memory-like NK cells with an increased ability to kill cancer cells while the cell proliferation thereof is maintained by priming with memory-like NK cells during a process of isolating peripheral blood mononuclear cells, starting the culturing for NK cell proliferation using the entire cells, and performing active proliferation, in order to overcome the disadvantage of memory-like NK cells that do not proliferate well. Through the process of removing CD3-positive T cells and culturing high-purity NK cells without prey cells, there is an effect capable of proliferating a sufficient number of memory-like NK cells suitable for patient administration.

In an aspect, the present invention relates to memory-like NK cells produced by the production method of the present invention.

In an embodiment, the memory-like NK cells produced by the production method of the present invention may have increased expression of CD94 or CD25 compared to NK cells and decreased expression of NKp80 compared to NK cells.

In an embodiment, the memory-like NK cells produced by the production method of the present invention may have increased cell proliferation rate compared to NK cells.

The memory-like NK cells produced by the production method of the present invention have an excellent ability to kill cancer cells compared to NK cells and thus may be used as an immune cell therapeutic agent, and have a memory-like ability and thus have the ability to be activated again and proliferate again when a second stimulus is applied, and have the advantage of being able to survive for 2 months or longer in vivo.

In an aspect, the present invention relates to a pharmaceutical composition for preventing or treating cancer comprising the memory-like NK cells of the present invention as an active ingredient.

In an aspect, the present invention relates to an immune cell therapeutic agent for treating cancer comprising the memory-like NK cells of the present invention as an active ingredient.

In an embodiment, the cancer may be any one or more selected from the group consisting of brain tumor, melanoma, myeloma, non-small cell lung cancer, oral cancer, liver cancer, stomach cancer, colorectal cancer, breast cancer, lung cancer, bone cancer, pancreatic cancer, skin cancer, head or neck cancer, cervical cancer, ovarian cancer, colon cancer, small intestine cancer, rectal cancer, fallopian tube carcinoma, perianal cancer, endometrial carcinoma, vaginal carcinoma, vulvar carcinoma, Hodgkin's disease, esophageal cancer, lymph adenocarcinoma, bladder cancer, gallbladder cancer, endocrine adenocarcinoma, thyroid cancer, parathyroid cancer, adrenal cancer, soft tissue sarcoma, urethral cancer, penile cancer, prostate cancer, kidney or ureter cancer, renal cell carcinoma, renal pelvic carcinoma, central nervous system tumor, spinal cord tumor, brainstem gliomas and pituitary adenomas.

As used in the present invention, the terms “cancer” and “tumor” may be used interchangeably.

As used in the present invention, the term “prevention” refers to all actions that inhibit or delay the occurrence, development, and recurrence of cancer by administration of the composition according to the present invention.

As used in the present invention, the term “treatment” means all actions that improve or beneficially change the symptoms of cancer and its complications by administration of the composition according to the present invention. Those skilled in the art to which the present invention pertains will be able to determine the degree of improvement, enhancement and treatment by knowing the exact criteria of a disease for which the composition of the present invention is effective by referring to data presented by the Korean Academy of Medical Sciences, etc.

The therapeutically effective amount of the composition of the present invention may vary depending on many factors, for example, an administration method, a target site, a condition of a subject, and the like.

The pharmaceutical composition of the present invention is administered in a pharmaceutically effective amount. As used in the present invention, the term “pharmaceutically effective amount” refers to an amount enough to treat the disease at a reasonable benefit/risk ratio applicable to medical treatment and enough not to cause side effects. The effective dose level may be determined according to factors including the health condition of a subject, the type and severity of cancer, the activity of a drug, the sensitivity to a drug, an administration method, a time of administration, a route of administration, an excretion rate, duration of treatment, and drugs used in combination or simultaneously, and other factors well-known in the medical field. The composition of the present invention may be administered as an individual therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side effects by considering all the factors, which may be easily determined by those skilled in the art.

In an embodiment, the pharmaceutical composition may be one or more formulations selected from the group including oral formulations, external formulations, suppositories, sterile injection solutions and sprays.

The composition of the present invention may contain a carrier, a diluent, an excipient, or a combination of two or more thereof, which are commonly used in biological agents. The pharmaceutically acceptable carrier is not particularly limited as long as the carrier is suitable for in vivo delivery of the composition, and may be used by mixing, for example, compounds described in Merck Index, 13th ed., Merck & Co. Inc., saline, sterile water, a Ringer's solution, buffered saline, a dextrose solution, a maltodextrin solution, glycerol, ethanol, and one or more of these components, and if necessary, other conventional additives such as an antioxidant, a buffer, and a bacteriostat may be added. In addition, the pharmaceutical composition may be prepared in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be prepared preferably according to each disease or ingredient using as a suitable method in the art or a method disclosed in Remington's Pharmaceutical Science (Mack Publishing Company, Easton PA, 18th, 1990).

The composition of the present invention may further contain one or more active ingredients exhibiting the same or similar functions. The composition of the present invention includes 0.0001 to 10 wt %, preferably 0.001 to 1 wt % of the protein, based on the total weight of the composition.

The pharmaceutical composition of the present invention may further contain pharmaceutically acceptable additives. At this time, the pharmaceutically acceptable additive may be used with starch, gelatinized starch, microcrystalline cellulose, lactose, povidone, colloidal silicon dioxide, calcium hydrogen phosphate, lactose, mannitol, syrup, arabic gum, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, sucrose, dextrose, sorbitol, talc, and the like. The pharmaceutically acceptable additive according to the present invention is preferably contained in an amount of 0.1 part by weight to 90 parts by weight based on the composition, but is not limited thereto.

The composition of the present invention may be administered parenterally (for example, intravenously, subcutaneously, intraperitoneally, or topically) or orally depending on a desired method, and most preferably orally administered. The dose range thereof varies according to the weight, age, sex, health condition, diet, administration time, administration method, and excretion rate of a subject, the severity of a disease, etc.

Liquid formulations for oral administration of the composition of the present invention correspond to suspensions, internal solutions, emulsions, syrups, etc., and may include various excipients, such as wetting agents, sweeteners, fragrances, preservatives, and the like, in addition to water and liquid paraffin, which are commonly used simple diluents. Formulations for parenteral administration include sterilized aqueous solutions, non-aqueous solvents, suspensions, emulsions, lyophilized agents, suppositories, and the like.

In an aspect, the present invention relates to an anticancer adjuvant for enhancing the activity of an anticancer agent including the memory-like NK cells as an active ingredient.

In an embodiment, the anticancer agent may be eribulin, carboplatin, cisplatin, Halaven, 5-fluorouracil (5-FU), gleevec, Vincristine, Vinblastine, Vinorelvine, Paclitaxel, Docetaxel, Etoposide, Topotecan, Irinotecan, Dactinomycin, Doxorubicin, Daunorubicin, valrubicin, flutamide, gemcitabine, Mitomycin, or Bleomycin.

In an aspect, the present invention relates to a method of treating liver cancer, comprising administering the pharmaceutical composition or the cell therapeutic agent to a subject.

In the present invention, the “subject” is not particularly limited as long as the subject is any subject for the purpose of preventing or treating cancer, and includes animals including humans, for example, mammals including non-primates (e.g., cow, pig, horse, cat, dog, rats and mice) and primates (e.g., monkey such as cynomolgous monkeys and chimpanzee).

The pharmaceutical composition or the cell therapeutic agent of the present invention may be administered in a therapeutically effective amount or pharmaceutically effective amount.

As used in the present invention, the term “therapeutically effective amount” means an amount of a pharmaceutically acceptable salt of the composition effective for preventing or treating a target disease, and the therapeutically effective amount of the composition of the present invention may vary depending on many factors, such as a method of administration, a target site, the condition of a patient, and the like. Accordingly, when used in the human body, the dose should be determined as an appropriate amount in consideration of both safety and efficiency. It is also possible to estimate the amount used in humans from the effective amount determined through animal experiments. These matters to be considered when determining the effective amount are described in, for example, Hardman and Limbird, eds., Goodman and Gilman's The Pharmacological Basis of Therapeutics, 10th ed. (2001), Pergamon Press; and E. W. Martin ed., Remington's Pharmaceutical Sciences, 18th ed. (1990), Mack Publishing Co.

As used in the present invention, the term “pharmaceutically effective amount” refers to an amount enough to treat the disease at a reasonable benefit/risk ratio applicable to medical treatment and enough not to cause side effects. The effective dose level may be determined according to factors including the health condition of a patient, the type and severity of a disease, the activity of a drug, the sensitivity to a drug, an administration method, a time of administration, a route of administration, an emission rate, duration of treatment, and combined or simultaneously used drugs, and other factors well-known in the medical field. The composition of the present invention may be administered as an individual therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side effects by considering all the factors, which may be easily determined by those skilled in the art.

### MODE OF THE INVENTION

Hereinafter, the present invention will be described in more detail through Examples. However, these Examples are more specifically illustrative the present invention, and the scope of the present invention is not limited to these Examples.

### Example 1. Production of Memory-Like NK Cells

### 1-1. Flask Coating

One day before blood collection, a solution of 5 to 10 μg/ml of fibronectin (FC010, Sigma), 1 to 2 mg/ml of γ-globulin (Green Cross), and 5 to 10 μg/ml of anti-CD56 antibody (559043, BD Biosciences) diluted in DPBS was added to a flask to be used in advance, spread evenly, and coated at 2 to 8° C. for 16 hours or more. At this time, if the size of the flask was 25 cm², the flask was coated with a total of 3 to 5 ml of the solution, and if the size of the flask was 75 cm², the flask was coated with 5 to 10 ml of the solution. The remaining coating solution was removed from the coated flask, washed with 10 ml of DPBS, and then DPBS was removed.

### 1-2. Isolation of Peripheral Blood Mononuclear Cells (PBMC)

Blood was collected from the veins of a total of 5 healthy donors into heparin-coated vacuum blood collection tubes (367874, BD) respectively, and the blood was carefully transferred to a 50 ml tube containing 15 ml of Histopaque 1077 (10771, Sigma-Aldrich). The tube containing the blood was centrifuged at 1000×g at room temperature for 10 minutes in a break off state, the tube was carefully taken out, and then the supernatant was transferred to a new 50 ml tube, added in a water bath set at 56° C., inactivated for 30 minutes, and centrifuged at 1400×g for 10 minutes. The tube was taken out and only the supernatant was added in a new 50 ml tube to complete autologous plasma isolation. The autologous plasma was refrigerated (2 to 8° C.) and used. The plasma was isolated, and a mononuclear cell layer was carefully isolated from the remaining tube, transferred to a new 50 ml tube, fully filled with DPBS (17-512F, Lonza), and centrifuged at 910×g for 10 minutes. After centrifugation, the supernatant was discarded, and the deposited cells were resuspended in 30 ml of DPBS and centrifuged at 910×g for 10 minutes. After the supernatant was discarded, the deposited cells were resuspended in 5 ml of a red blood cell lysis buffer (00-4333-57, Thermo Fisher Scientific) and then left at room temperature for 5 minutes. Thereafter, the cell suspension was added with 30 ml of DPBS to terminate the reaction, and then centrifuged at 910×g for 10 minutes, and the supernatant was removed. The deposited cells were resuspended in 10 ml of DPBS and then centrifuged at 910×g for 10 minutes. The supernatant was discarded and the remaining deposited cells were resuspended in 10 ml of a culture medium (NK MACS medium, 130-114-429, Miltenyi biotec), and then a small amount of cells were taken, diluted 10-fold, and counted.

### 1-3. Primary Culture of NK Cells

The cell suspension prepared in Example 1-2 was taken and added in the 25 cm² coated flask prepared in Example 1-1, added with 2 to 10% (v/v) of autologous plasma, 0.1 to 1 μg/ml of anti-NKp46 antibody (130-094-271, Miltenyi Biotec), 100 to 1000 IU/ml of IL-2 (146545, Boehringer Ingelheim) and 5 to 100 ng/ml of IL-18 (9124IL-500-CF, R&D Systems), and cultured in an incubator at 37° C. and 5% CO₂ until the cell density reached 80% or more. 2.5 ml of a fresh medium containing 10% autologous plasma, 100 to 1000 IU/ml of IL-2, 5 to 100 ng/ml of IL-18, and 0.1 to 1 μg/ml of anti-NKp46 antibody was added to the flask during the cell culturing and then continuously cultured until the cell density reached 80% or more. Cells attached to the bottom of a 25 cm² flask where the cell culture was completed were detached using a cell scraper, and then 7.5 ml of the cell suspension was transferred to a 75 cm² flask. 8 ml of a fresh medium containing 2 to 10% of autologous plasma and 100 to 1,000 IU/ml of IL-2 was added thereto, and then further continuously cultured until the cell density reached 80% or more. 15 ml of a fresh medium containing 2 to 10% of autologous plasma and 100 to 1,000 IU/ml of IL-2 was added to the flask during the cell culturing, and then further cultured until the cell density reached 80% or more. The cells attached to the bottom of the flask where the cell culture was completed were detached using a cell scraper, and then 30 ml of the cell suspension was transferred to a tube and centrifuged at 910×g for 10 minutes.

### 1-4. Stimulation of Memory-Like NK Cells

The supernatant of the cells centrifuged in Example 1-3 was removed, resuspended in 10 ml of DPBS, and then centrifuged at 910×g for 10 minutes to wash the cells. The washing was repeated twice. The washed cells were resuspended with 30 ml of a fresh medium containing 2 to 10% (v/v) of autologous plasma, 2 to 50 ng/ml of IL-12, 1 to 10 ng/ml of IL-15, and 10 to 100 ng/ml of IL-18, added in a new 75 cm² flask, and cultured and stimulated for 12 to 16 hours. Cells stimulated for 12 to 16 hours were collected, transferred to a 50 ml tube, and centrifuged at 910×g for minutes. The supernatant was removed, resuspended in 10 ml of DPBS, and then centrifuged again at 910×g for 10 minutes and washed. The washing was repeated twice.

### 1-5. Secondary Culture of NK Cells

The cells washed in Example 1-4 above were resuspended with 30 ml of a fresh medium containing 2 to 10% (v/v) of autologous plasma, 100 to 1000 IU/ml of IL-2, and 1 to 10 ng/ml of IL-15 and then cultured in a new 75 cm² flask until the cell density reached 80% or more. When the cell density in the flask reached 80% or more, the cells were transferred to a 175 cm² flask, added with a fresh medium containing 100 to 1000 IU/ml of IL-2, and continuously cultured to produce memory-like NK cells.

### Example 2. NK Cell Purity Analysis

To measure the purity of memory-like NK cells produced in Example 1, 1 to 5×10⁵ cells were taken per sample and the cell suspension was added in a 1.5 ml tube, added with 1 ml of DPBS, mixed well, and centrifuged at 910×g for 5 minutes. The supernatant was removed and repeatedly washed twice using 500 μl of DPBS. The washed cell deposit was resuspended in 98 μl of an FACS staining buffer (554657, BD Biosciences), added with 2 μl of a human FcR blocking reagent (130-059-901, Miltenyi Biotec), mixed well, and reacted for 20 minutes in the dark at 0 to 4° C. Thereafter, the washing was repeated three times using 500 μl of DPBS, and the washed cell deposit was resuspended in 94 μl of an FACS staining buffer, and then added with 2 μl of anti-CD3 antibody (48-0038-82, eBioscience), anti-CD56 antibody (318303, BioLegend) and anti-CD19 antibody (555415, BD Biosciences), mixed well, and reacted for 30 minutes in the dark at 0 to 4° C. The washing was repeated three times using 500 μl of DPBS, the washed cell deposit was resuspended in 200 μl of an FACS staining buffer, and then purity analysis of CD3-negative and CD56-positive NK cells was performed using a flow cytometer. Further, a distribution map of day 0 was obtained from the results of analyzing the distribution of NK cells immediately after obtaining peripheral blood mononuclear cells by collecting blood from 5 donors, and the distribution of memory-like NK cells (12 to 15 days) finally obtained through the process of Example 1 was analyzed. A control group was used with cells in which peripheral blood mononuclear cells were cultured in a culture medium containing 2 to 10% (v/v) of autologous plasma and 1 ng/ml of IL-15.

TABLE 1
NK cell distribution (%)

12-15 Day

| | 0 Day | Control | Memory like NK cells |
|---|---|---|---|
| Donor 1 | 30.0 | 20.3 | 71.6 |
| Donor 2 | 18.4 | 53.65 | 94.3 |
| Donor 3 | 21.6 | 32.7 | 92.2 |
| Donor 4 | 19.7 | 32.7 | 86.9 |
| Donor 5 | 41.57 | 44.0 | 92.38 |

Table 1 above shows the distribution of NK cells obtained from a total of 5 healthy donors. The distribution map of day 0 was obtained from the results of analyzing the distribution of NK cells immediately after obtaining peripheral blood mononuclear cells by collecting blood. After the culture for 12 to 15 days was completed, the distribution of NK cells was analyzed and the distribution of the final NK cells obtained was shown. CD3 negative and CD56 positive cells were considered as NK cells. Control was a negative control and corresponded to cells in which peripheral blood mononuclear cells were cultured in a culture medium containing 2 to 10% autologous plasma and 1 ng/ml of IL-15. As a result, it was found that the memory-like NK cells were significantly increased through the method of the present invention (Table 1 and FIG. 1).

### Example 3. Analysis of Anticancer Activity of NK Cells

The activity of the memory-like NK cells produced in Example 1 was measured. Specifically, a target human cancer cell line, K562 was washed twice repeatedly using its culture medium, complete IMDM (IMDM containing 10% FBS), the washed cell deposit was resuspended in 1 ml of complete IMDM, and then Calcein-AM (C1430, Invitrogen) was added thereto at a concentration of 0.01 μM, mixed well, and stained for 10 minutes in the dark at 37° C. The tube containing the stained K562 cells was centrifuged at 910×g for 5 minutes, the supernatant was removed, and then the tube was washed three times with an NK cell culture medium. NK cells were washed twice with the NK cell culture medium and then added in a 12-well plate together with Calcein-AM-stained K562 at a ratio of 5:1 (5×10⁵ NK cells and 1×10⁵ K562 cells per sample), and co-cultured for 4 hours in an incubator at 37° C. and a 5% CO₂ concentration. At this time, 1 ml of the culture medium containing 2 to 10% (v/v) of autologous plasma and 100 to 1,000 IU/ml of IL-2 was added to each well. The cells were recovered from the plate, transferred to a 1.5 ml tube, and centrifuged at 910×g for 5 minutes. The supernatant was removed, the tube was washed twice with 500 μl of DPBS, resuspended in 99 μl of an FACS staining buffer, and then added with 1 μl of a 7-AAD viability staining solution (420403, BioLegend), mixed well and stained for 5 to 10 minutes in the dark at room temperature. The stained cells were analyzed using a flow cytometer, and among the Calcein-AM positive cells, the 7-AAD positive cells were considered as cells killed by NK cells. In addition, a negative control group was used with cells cultured in a culture medium containing 2 to 10% (v/v) of autologous plasma and 1 ng/ml of IL-15.

TABLE 2
Cytotoxicity (%)

| | Control | Memory-like NK cells |
|---|---|---|
| Donor 1 | 3.8 | 65.8 |
| Donor 2 | 41.3 | 69.7 |
| Donor 3 | 37.5 | 67.7 |
| Donor 4 | 50.0 | 70.2 |
| Donor 5 | 30.5 | 63.0 |

Table 2 above showed the cancer cell killing ability (hereinafter “cytotoxicity”) of memory-like NK cells finally obtained by culturing from a total of 5 healthy donors. A negative control group was used with cells cultured in a culture medium containing 2 to 10% (v/v) of autologous plasma and 1 ng/ml of IL-15. As a result, it was shown that the cytotoxicity of an experimental group (memory-like NK cells) was significantly increased compared to the negative control group in cells from all donors (Table 2 and FIG. 2).

### Example 4. Expression Analysis of Identification Markers of Memory-Like NK Cells

Compared to normal NK cells, the memory-like NK cells had increased expression of CD94 and CD25 and decreased expression of NKp80 (Margery Gang, et al., Semin Hematol. 57 (4): 185-193, 2020), and thus the expression of memory-like NK cells-specific identification markers was analyzed in the memory-like NK cells produced in Example 1. Specifically, negative control cells cultured for 12 to 15 days by adding 2 to 10% (v/v) of autologous plasma and 1 ng/ml of IL-15 to the culture medium, and the memory-like NK cells produced by the method of Example 1 above for 12 to 15 days were harvested, 5×10⁵ cells per sample were taken and washed three times with DPBS, added with 98 μl of an FACS staining buffer and 2 μl of a human FcR blocking reagent, mixed well, and reacted at 0 to 4° C. for 20 minutes. After reaction, washing was repeated three times using 500 μl of DPBS, and the washed cell deposit was resuspended in 90 μl of an FACS staining buffer, and then added with 2 μl each of anti-CD3 antibody (48-0038-82, eBioscience), anti-CD56 antibody (47-0567-42, eBioscience), anti-CD94 antibody (305508, BioLegend), anti-CD25 antibody (562661, BD Biosciences), and anti-NKp80 antibody (130-125-238, Miltenyi Biotech), mixed well, and then reacted at 0 to 4° C. for 30 minutes. After the reaction was completed, washing was repeated three times using 500 μl of DPBS, the washed cell deposit was resuspended in 200 μl of an FACS staining buffer, and CD3-negative and CD56-positive NK cells were selected using a flow cytometer and then the expression level of each identification marker was analyzed.

FIG. 3 shows the results of comparing the receptor expression on memory-like NK cells compared to the negative control group, and it was shown that in the memory-like NK cells, CD94 and CD25 were increased and NKp80 was decreased (FIG. 3).

### Example 5. Analysis of Proliferation Ability of NK Cells

5-1. Comparison of the Number of Produced Memory-Like NK Cells with Entire Cells

In order to confirm the proliferation ability of the memory-like NK cells produced in Example 1, to compare the total number of cells finally harvested compared to the total number of cells from which the culture was first started, the starting day of the culture was set as day 0, and then the total number of cells was counted for each day while the culture progressed, and then a cumulative proliferation rate was calculated to determine how many cells had proliferated compared to day 0. In addition, compared to a negative control, which was cultured for 12 to 15 days by adding 2 to 10% (v/v) of autologous plasma and 1 ng/ml of IL-15 to the culture medium, the overall cell proliferation level of the memory-like NK cells produced in Example 1 of the present invention was confirmed.

TABLE 3
Overall cell proliferation level (n)

| | 0 Day | 6 Day | 9 Day | 12 Day |
|---|---|---|---|---|
| Donor 1 | 1.0 | 2.6 | 10.4 | 65.9 |
| Donor 2 | 1.0 | 5.9 | 23.2 | 189.4 |
| Donor 3 | 1.0 | 3.0 | 10.9 | 35.6 |
| Donor 4 | 1.0 | 3.3 | 14.0 | 45.9 |
| Donor 5 | 1.0 | 3.2 | N/A | 48.6 |

TABLE 4
Overall cell proliferation level of experimental
group compared to negative control (0 day/12 day)

| | Control | Memory-like NK cells |
|---|---|---|
| Donor 1 | 2.4 | 65.9 |
| Donor 2 | 1.2 | 189.4 |
| Donor 3 | 18.0 | 35.6 |
| Donor 4 | 11.5 | 45.9 |
| Donor 5 | 0.1 | 48.6 |

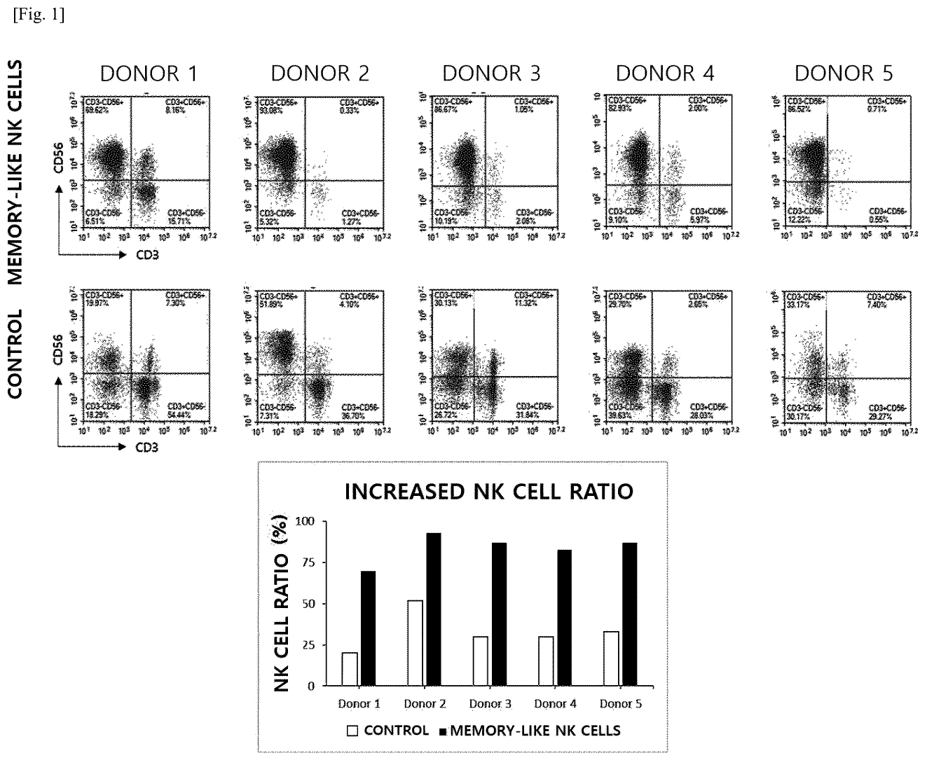

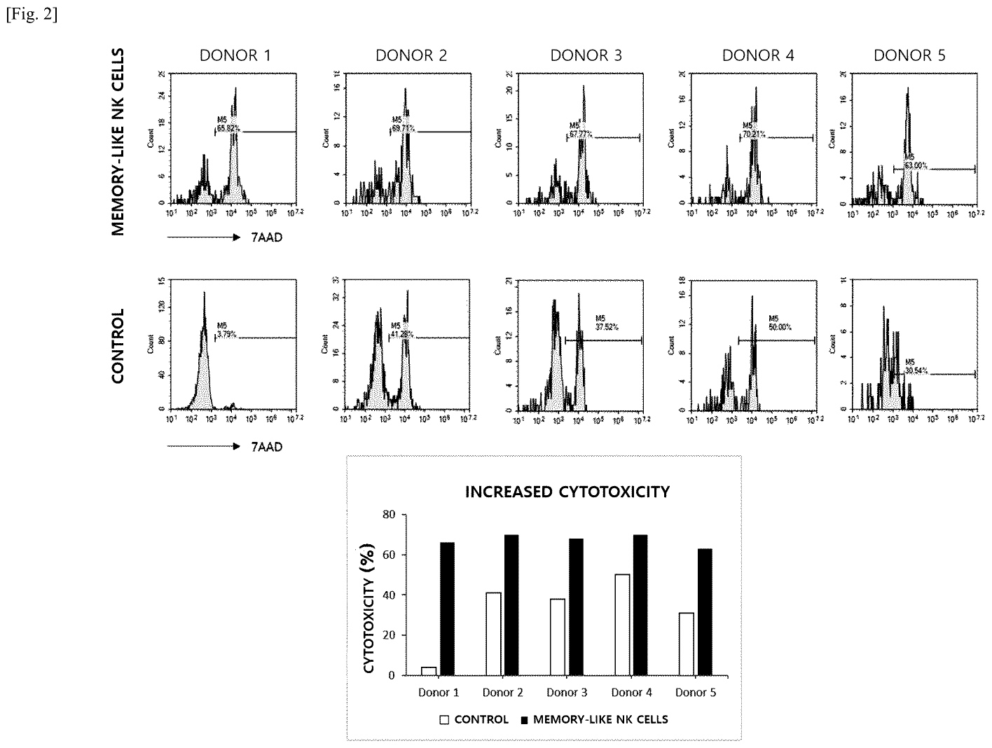

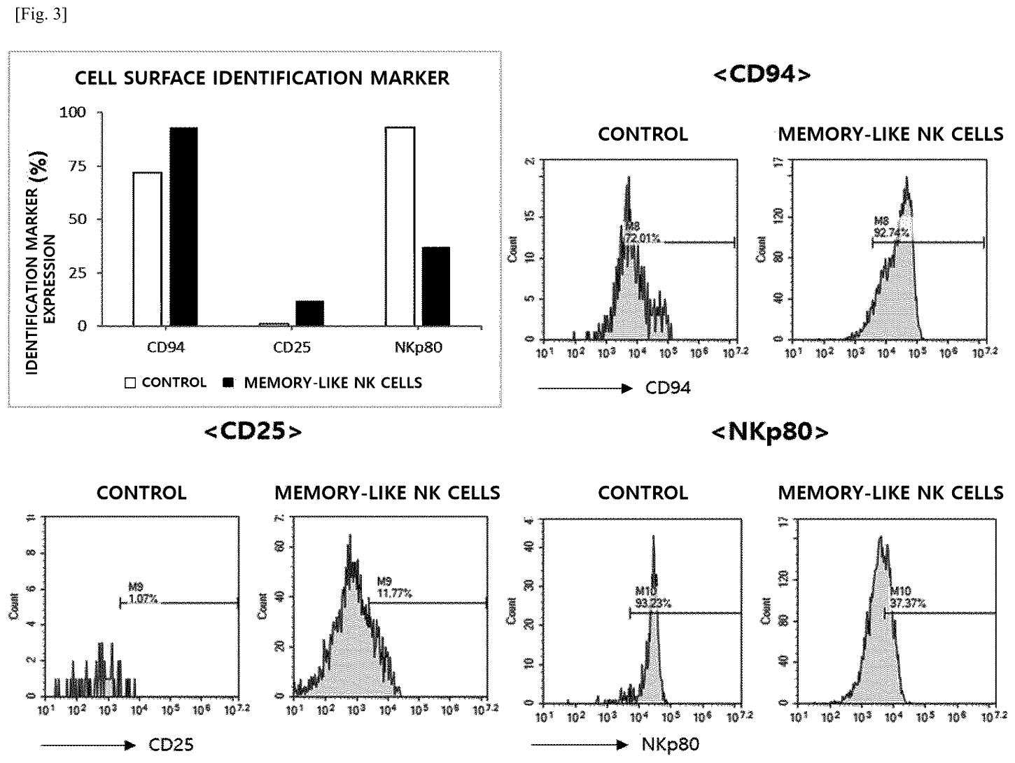

As a result, it was shown that although there was some variation depending on a donor, cells proliferated at least 35-fold and on average 77-fold compared to the start date of culture (Table 3 and FIG. 4). In addition, all the donors showed average 12-fold higher cell proliferation rate than the negative control group (Table 4 and FIG. 5).

5-1. Comparison of the Number of Produced Memory-Like NK Cells with the Number of NK Cells at the Start of Culture

In order to confirm the proliferation ability of the memory-like NK cells produced in Example 1, to compare the total number of cells finally harvested compared to the number of NK cells at the start of the culture, the number of NK cells that proliferated during the culture was calculated based on the staring day (day 0) of the culture, and how much a several fold of cells had proliferated was analyzed. In addition, compared to a negative control, which was cultured for 12 to 15 days by adding 2 to 10% (v/v) of autologous plasma and 1 ng/ml of IL-15 to the culture medium, the NK cell proliferation level of the memory-like NK cells (experimental group) produced in Example 1 of the present invention was confirmed.

TABLE 5
Proliferation rate of NK cells compared to initial cultured NK cells

| | 0 Day | 6 Day | 9 Day |
|---|---|---|---|
| Donor 1 | 1.0 | 3.7 | 153.1 |
| Donor 2 | 1.0 | 20.3 | 956.0 |
| Donor 3 | 1.0 | 7.3 | 143.2 |
| Donor 4 | 1.0 | 8.4 | 193.7 |
| Donor 5 | 1.0 | 5.6 | 98.0 |

TABLE 6
Proliferation rate of final NK cells compared
to initial cultured NK cells (0 day/12 day)

| | Control | Memory-like NK cells |
|---|---|---|
| Donor 1 | 1.6 | 153.1 |
| Donor 2 | 3.4 | 956.0 |
| Donor 3 | 25.2 | 143.2 |
| Donor 4 | 17.4 | 193.7 |
| Donor 5 | 0.1 | 98.0 |

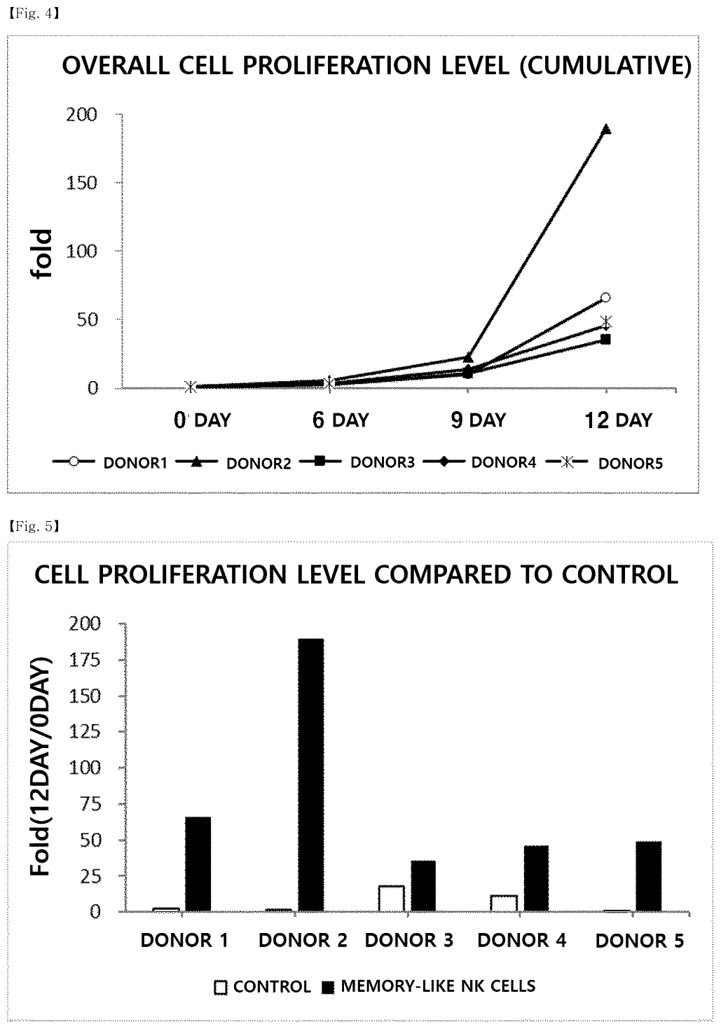

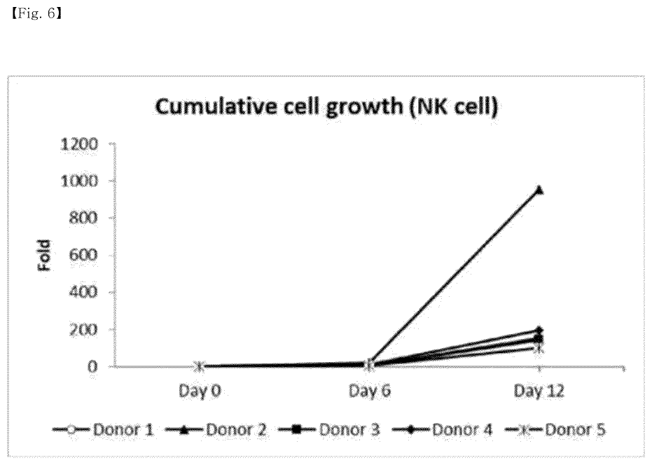

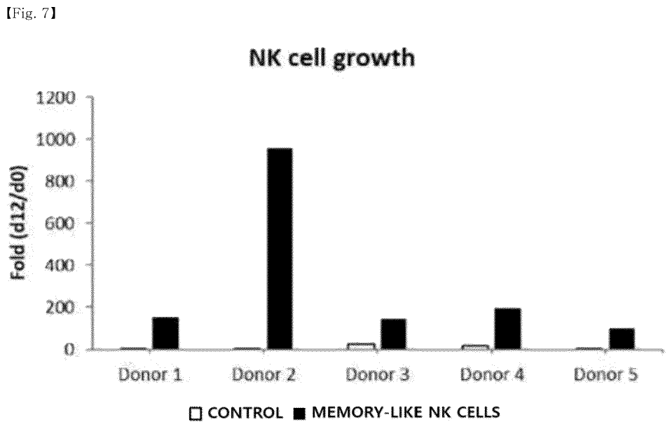

As a result, there was variation depending on the donor, but NK cells were found to be proliferated at least 98-fold and on average 309-fold (Table 5 and FIG. 6). In addition, in all donors, the negative control group showed an average 9.5-fold NK cell proliferation rate, while the experimental group showed an average 308.8-fold NK cell proliferation rate (approximately 32-fold), and thus it was shown that NK cells were significantly proliferated in the experimental group compared to the negative control group (Table 6 and FIG. 7).

1. A method for producing memory-like NK cells comprising:
1) isolating peripheral blood mononuclear cells (PBMC) from peripheral blood;
2) culturing by treating plasma, anti-NKp46 antibody, IL-2, and IL-18;
3) transferring the cells to a new culture vessel and culturing by treating the cells with plasma and IL-2;
4) transferring the cells to a new culture vessel and culturing by treating the cells with plasma, IL-12, IL-15, and IL-18; and
5) transferring the cells to a new culture vessel and culturing by treating the cells with plasma, IL-12, and IL-15.

2. The method of claim 1, wherein in step 2), the cells are cultured in a culture vessel coated with fibronectin, γ-globulin, and anti-CD56 antibody.

3. The method of claim 1, wherein in step 2), the cells are treated with 1 to 20% (w/w) of the plasma, 0.01 to 10 μg/ml of the anti-NKp46 antibody, 10 to 5000 IU/ml of IL-2, and 2 to 500 ng/ml of IL-18.

4. The method of claim 1, wherein step 2) is performed twice or more.

5. The method of claim 1, wherein in step 3), the cells are treated with 1 to 20% (w/w) of the plasma and 10 to 5000 IU/ml of IL-2.

6. The method of claim 1, wherein step 3) is performed twice or more.

7. The method of claim 1, wherein in step 4), the cells are treated with 1 to 20% (w/w) of the plasma, 0.5 to 200 ng/ml of IL-12, 0.1 to 100 ng/ml of IL-15, and 2 to 500 ng/ml of IL-18.

8. The method of claim 1, wherein the culturing in step 4) is performed for 10 to 20 hours.

9. The method of claim 1, wherein in step 5), the cells are treated with 1 to 20% (w/w) of the plasma, 10 to 5000 IU/ml of IL-2, and 0.1 to 100 ng/ml of IL-15.

10. The method of claim 1, further comprising:
culturing by treating the cells with a fresh medium containing IL-2, after step 5).

11. The method of claim 10, wherein 10 to 5000 IU/ml of IL-2 is treated.

12. Memory-like NK cells produced by the method of claim 1.

13. The cells of claim 12, wherein the expression of CD94 or CD25 is increased compared to NK cells.

14. The cells of claim 12, wherein the expression of NKp80 is decreased compared to NK cells.

15. The cells of claim 12, wherein the cell proliferation rate is increased compared to NK cells.

16. A pharmaceutical composition for preventing or treating cancer comprising the memory-like NK cells of claim 12 as an active ingredient.

17. The composition of claim 16, wherein the cancer is any one or more selected from the group consisting of brain tumor, melanoma, myeloma, non-small cell lung cancer, oral cancer, liver cancer, stomach cancer, colorectal cancer, breast cancer, lung cancer, bone cancer, pancreatic cancer, skin cancer, head or neck cancer, cervical cancer, ovarian cancer, colon cancer, small intestine cancer, rectal cancer, fallopian tube carcinoma, perianal cancer, endometrial carcinoma, vaginal carcinoma, vulvar carcinoma, Hodgkin's disease, esophageal cancer, lymph adenocarcinoma, bladder cancer, gallbladder cancer, endocrine adenocarcinoma, thyroid cancer, parathyroid cancer, adrenal cancer, soft tissue sarcoma, urethral cancer, penile cancer, prostate cancer, kidney or ureter cancer, renal cell carcinoma, renal pelvic carcinoma, central nervous system tumor, spinal cord tumor, brainstem gliomas and pituitary adenomas.

18-19. (canceled)

20. An anticancer adjuvant for enhancing the activity of an anticancer agent comprising the memory-like NK cells of claim 12 as an active ingredient.

21. The adjuvant of claim 20, wherein the anticancer agent is eribulin, carboplatin, cisplatin, Halaven, 5-fluorouracil (5-FU), gleevec, Vincristine, Vinblastine, Vinorelvine, Paclitaxel, Docetaxel, Etoposide, Topotecan, Irinotecan, Dactinomycin, Doxorubicin, Daunorubicin, valrubicin, flutamide, gemcitabine, Mitomycin, or Bleomycin.

22-23. (canceled)

24. A method of preventing or treating cancer, the method comprising administering to a subject in need thereof the memory-like NK cells of claim 12 as an active ingredient.
